(19) United States
(12) Patent Application Publication
(10) Pub. No.: US 2009/0221525 A1
(43) Pub. Date: Sep. 3, 2009

(54) FUNCTIONAL FOODS AGAINST TUMORS

(76) Inventors: Johannes F. Coy, Grossostheim (DE); Stephan Hausmanns, Wiesbaden (DE); Jorg Kowalczyk, Eisenberg/Steinborn (DE)

Correspondence Address: OSTROLENK FABER GERB & SOFFEN, 1180 AVENUE OF THE AMERICAS, NEW YORK, NY, 100368403, US

(21) Appl. No.: 12/282,536

(22) PCT Filed: Mar. 16, 2007
(86) PCT No.: PCT/EP07/02321

§ 371 Date: Jan. 16, 2009

(30) Foreign Application Priority Data
Mar. 21, 2006 (DE) 10 2006 014 543.7

(51) Int. Cl. A61K 31/715

(52) U.S. Cl. 514/53; 514/54

(57) ABSTRACT

The present invention relates to the technical field of functional foods, more precisely novel functional foods for the treatment and/or prevention of malignant carcinomas of the human or animal body, and also to processes for producing such functional foods.

The present invention relates to the technical field of functional foods, more precisely novel functional foods for the treatment and/or prevention of malignant carcinomas of the human or animal body, and also to processes for producing such functional foods.

Nutrition and diseases are connected. For instance, it has long been known, for example that adult-onset diabetes (diabetes type II) occurs especially in people who consume a carbohydrate-rich diet excessively. In addition, there are indications that the occurrence of certain cancers is directly connected to certain nutritional habits. For example, repeated and excessive intake of fructose in an animal experiment (rats) leads to increased growth of chemically induced liver tumors (Enzmann H. et al. (1989) Carcinogenesis 10:1247-1252). Also in humans, the consumption of fructose appears to be connected to the occurrence of various cancers (Michaud D. S. et al. (2002) J. Natl. Cancer Inst. 94: 1293-1300; Higginbotham H. et al. (2004) J. Natl. Cancer Inst. 96: 229-233). Also, unbalanced carbohydrate-rich nutrition appears to be connected to the occurrence of Alzheimer's disease (Henderson S. T. (2004) Med. Hypotheses 62: 689-700).

Accordingly, in the interim, certain diets are proposed for the prevention and treatment of diseases. For instance, it is known that a ketogenic diet, that is to say a diet which is based on avoiding carbohydrate fractions in food, in individual case studies beneficially affects the course of disease in carcinomas (Nebeling L. C. et al. (1995) J. Am. Coll. Nutr. 14:202-208).

Unbalanced diets are known which entirely omit carbohydrate supply. Such diets avoid glucose-containing food components or those food components from which glucose can be cleaved enzymatically in the body. The energy metabolism of the majority of healthy body cells is usually based exclusively on the utilization (combustion or fermentation) of glucose. For instance, the brain of a human requires about 25% of an individual's total energy requirement, wherein this can be satisfied exclusively by glucose. If glucose is absent in the diet, the body mobilizes glucose from the breakdown of storage fats (lipolysis), but also from the breakdown of vital and structure-determining proteins. The process is termed “ketogenesis”; the sequence loss of functions, coma and death occurs.

For over 80 years it has already been suspected that the cells of malignant tumors have an altered glucose metabolism (Warburg O. et al. (1924) Biochem Z. 152:309-344). Accordingly, it is known that certain tumor cells, even in the presence of oxygen, ferment glucose to lactic acid (lactate) (Warburg effect). On the other hand, certain healthy tissue cells also exhibit glucose fermentation metabolism (aerobic glycolysis), for example retina and testis cells.

In the light of the prior art, the technical problem underlying the present invention is to provide functional foods and processes for production thereof and also treatment processes, by which the occurrence of malignant tumors in the human or animal body can be prevented or their growth suppressed or brought to a stop.

The underlying technical problem is solved by providing a process in which a low glycemic food composition, that is to say a food or a food component having a low glycemic index (GI) or a composition of a plurality thereof, is used for producing a functional food which serves for treating carcinomas of the human or animal body and/or preventing malignant carcinomas.

The invention therefore envisages providing, by the use of a low glycemic food composition, a functional food which has a low glycemic index. This ensures that the malignant tumor cells which are dependent on aerobic glucose fermentation for their energy metabolism no longer receive a sufficient energy supply because of the lack of highly glycemic components. An occurrence of malignant tumor cells is thus prevented; already existing malignant tumor cells are inhibited in their growth or die.

In the context of the present invention, a “low glycemic food composition” is taken to mean one or more foods or one or more food components and compositions thereof which have a low glycemic index (GI). A low glycemic index in the present case is taken to mean a GI of 50 or less. Preferably, the GI is 40 or less, particularly preferably 30 or less, or 20 or less. The invention envisages in this case that the GI is not below a limiting value. That means, preferably, the low glycemic food composition comprises at least one glucose source such as sucrose, maltose, starch/amylose, lactose or glucose. Preferably, the GI is at least 2 or more, at least 5 or more, or at least 10 or more.

The inventors have found, surprisingly, that for the metabolic pathway which mediates aerobic glucose fermentation in certain malign tumor cells is based on a certain enzyme, transketolase-like 1 (TKTL-1). Essentially, in malign tumor cells, up-regulation of the TKTL-1 mRNA (TKTL-1 expression) may be detected. In contrast thereto, the known aerobic glucose fermentation in healthy body cells is based on transketolase (TKT).

Among the malign tumor cells, surprisingly only a certain population operates such an aerobic glucose fermentation and obtains its energy exclusively therefrom. Without the supply of glucose, these tumor cells cannot maintain their energy balance. The use according to the invention is particularly suitable for those patients in which at least one cell, that is to say particularly a malign tumor cell, has an altered glucose metabolism which is based on an altered activity of the enzyme TKTL-1. By means of suitable enzyme assays or by the detection of TKTL-1 expression, preferably at the mRNA level, in body cells, for example in the context of an examination for clinical diagnosis, the body of patients can be determined which is particularly suitable for treatment with the functional food according to the invention having a low glycemic food composition.

Accordingly, the present invention relates to the use of a low glycemic food composition for producing a functional food for the treatment and/or prevention of malignant carcinomas of an animal or human body, wherein this has at least one body cell which has a glucose metabolism which is altered compared with the normal state, particularly an altered aerobic glucose fermentation metabolism. According to the invention, the normal state is defined in that the cell, in accordance with its physiological function and in accordance with its tissue type, has the metabolic pathway typical thereof for energy production from glucose. This means, especially, that in the normal state the body cell respires glucose primarily with reaction with oxygen. In special tissue types, the normal state of a cell can also be characterized by an aerobic glucose fermentation metabolism. Such cells are, for example, known from the retina or the testes. Such cells have a transketolase activity in the normal state.

In contrast, an altered glucose metabolism, in particular an altered aerobic glucose fermentation metabolism, is especially characterized in that the body cell has an activity or concentration of the enzyme transketolase-like 1 (TKTL-1) which is altered compared with the normal state. According to the invention, this is also taken to mean those cells which, in a TKTL-1 enzyme assay, exhibit altered enzyme activity and/or on determination of TKTL-1 gene expression, preferably at the mRNA level, in a manner known per se, exhibit an altered content of the gene product. Preferably, the altered activity is taken to mean an increased enzyme activity. Preferably, an altered content of the TKTL-1 gene product is taken to mean the occurrence of the TKTL-1 gene product in cells which in the normal state do not express or produce TKTL-1 and/or have an increased fraction of the TKTL-1 gene product.

A low glycemic food, food component and/or food composition used according to the invention for preparing a functional food preferably has at least one carbohydrate source selected from monosaccharides, disaccharides and polysaccharides and also from monosaccharide alcohols, disaccharide alcohols and polysaccharide alcohols. The low glycemic food composition has at least one low glycemic or non-glycemic carbohydrate source or component. According to the invention, preferably, the low glycemic food composition, in addition to the at least one low glycemic or non-glycemic carbohydrate source or carbohydrate component, has at least one (additional) high glycemic carbohydrate source or carbohydrate component. Hereinafter, the preferred constituents or components of the low glycemic food composition used according to the invention are described in more detail.

Preference is given to glucose-yielding low glycemic sucrose isomers. Particularly preferably, the low glycemic food composition, as, preferably sole, carbohydrate source, contains isomaltulose (6-O-α-D-glucopyranosylfructose; Palatinose®), preferably in a fraction of 5 to 90% by weight, more preferably in a fraction of 25 to 75% by weight (based on total dry matter content).

Particularly preferably, the low glycemic food composition, as, preferably sole, carbohydrate source, contains leucrose, preferably in a fraction of 5 to 90% by weight, more preferably in a fraction of 25 to 75% by weight (based on total dry matter content).

Particularly preferably, the low glycemic food composition, as, preferably sole, carbohydrate source, contains trehalulose, preferably in a fraction of 5 to 90% by weight, more preferably in a fraction of 25 to 75% by weight (based on total dry matter content).

Particularly preferably, the low glycemic food composition, as, preferably sole, carbohydrate source, contains turanose, preferably in a fraction of 5 to 90% by weight, more preferably in a fraction of 25 to 75% by weight (based on total dry matter content).

Particularly preferably, palatinose, leucrose, tehalulose or turanose are used alone or in combination with other polyols, inulin, polydextrose or resistant starches.

Particularly preferably, the low glycemic food composition, as, preferably sole, carbohydrate source, contains resistant starch, preferably soluble starch, preferably in a fraction of 5 to 90% by weight, more preferably in a fraction of 25 to 75% by weight (based on total dry matter content). Alternatively, or in addition, insoluble starch is provided.

Particularly preferably, the low glycemic food composition, as, preferably sole, carbohydrate source, contains polydextrose, preferably in a fraction of 5 to 90% by weight, more preferably in a fraction of 25 to 75% by weight (based on total dry matter content).

The invention also relates to low glycemic food compositions in which the low glycemic sucrose isomer is the sole bulking sweetening agent present in the composition. In a further preferred embodiment, the invention relates to an abovementioned composition, wherein the low glycemic sucrose isomer is the sole sugar present in the mixture in the composition. In a further preferred embodiment, the invention relates to an abovementioned composition, wherein the low glycemic sucrose isomer is the sole sweetening agent present in the mixture composition.

Further preference is given to sugar alcohols. Preferably, the low glycemic food composition, as, preferably sole, carbohydrate source, contains at least one sugar alcohol selected from the group consisting of: isomalt, 1,1-GPM (1-O-α-D-glucopyranosyl-D-mannitol), 1,6-GPS (6-O-α-D-glucopyranosyl-D-sorbitol), 1,1-GPS (1-O-α-D-glucopyranosyl-D-sorbitol), lactitol, maltitol, erythritol, xylitol, mannitol, sorbitol, maltitol syrup, hydrogenated and non-hydrogenated starch hydrolyzates and a mixture of two or more thereof, preferably in a fraction of 5 to 90% by weight, more preferably in a fraction of 25 to 75% by weight (based on total dry matter content).

Particular preference is given to disaccharide alcohols. Particularly preferably, the low glycemic food composition contains, as, preferably sole, carbohydrate source, isomalt, a mixture of 1,1-GPM and 1,6-GPS, if appropriate having a fraction of 1,1-GPS, preferably in a fraction of 5 to 90% by weight, more preferably in a fraction of 25 to 75% by weight (based on total dry matter content).

Further preference is given to oligosaccharides such as fructooligosaccharides, short-chain or medium-chain β-D-fructans. Particularly preferably, the low glycemic food composition, as, preferably sole, carbohydrate source, contains inulin, preferably in a fraction of 5 to 90% by weight, more preferably in a fraction of 25 to 75% by weight (based on total dry matter content).

Particularly preferably, the low glycemic food composition, as, preferably sole, carbohydrate source, contains oligofructose, preferably in a fraction of 5 to 90% by weight, more preferably in a fraction of 25 to 75% by weight (based on total dry matter content).

The invention also relates to low glycemic food compositions in which the sugar alcohol is the sole bulking sweetening agent present in the composition. In a further preferred embodiment, the invention relates to an abovementioned composition, wherein the sugar alcohol is the sole sweetening agent present in the mixture in the composition.

Further preference is given to fructose; particularly preferably, the low glycemic food composition, as, preferably sole, carbohydrate source, contains fructose, preferably in a fraction of 5 to 90% by weight, more preferably in a fraction of 25 to 75% by weight (based on total dry matter content). In a preferred variant, the low glycemic food composition contains at least 40% by weight (based on the total dry matter content) fructose as sole carbohydrate source. The invention also relates to low glycemic food compositions in which fructose is the sole sugar present in the composition. The invention also relates to low glycemic food compositions in which fructose in the sole bulking sweetening agent present in the composition. In a further preferred embodiment, the invention relates to an abovementioned composition, wherein fructose is the sole sweetening agent present in the mixture in the composition.

Preferably, neither the low glycemic food composition used according to the invention, nor the functional food produced is carbohydrate free or low-carbohydrate. Rather, it is provided that the carbohydrate component present is low glycemic. Therefore, it is also provided that the low glycemic food composition contains at least one high glycemic component. A high glycemic component in the present case is taken to mean especially a high glycemic glucose source, from which, in particular, by enzymatic cleavage in the digestive and absorption process, initially glucose is released into the organism. The high glycemic component is preferably selected from: glucose-yielding disaccharides, in particular sucrose, maltose, lactose and trehalose and also maltodextrins, preferably resistant, soluble or insoluble, and also starch/amylose and white flour products.

Particularly preferably, the food composition, in addition to a low glycemic carbohydrate source, contains at least one high glycemic carbohydrate source. Preferably, the low glycemic carbohydrate source and the high glycemic carbohydrate source are present in the weight ratio 1:1, 2:1 or 1:2. Preferably, the low glycemic food composition contains the high glycemic carbohydrate source in a fraction of 20% by weight or more, preferably 10% by weight or more, or 5% by weight or more (based on total dry matter content).

In a preferred embodiment, the low glycemic food composition contains 10% by weight, preferably 5% by weight,

In addition it is preferably provided that the glycemic index of the low glycemic food composition is modified by at least one further component which, in a manner known per se, decreases, delays or prevents the absorption and/or cleavage of glucose from food components. Preferably, such a component is selected from proteins, fats or thickeners.

In a preferred embodiment, the low glycemic food composition contains at least 10% by weight, preferably at least 20% by weight, at least 30% by weight or at least 40% by weight (based on total dry matter content) of a fat component selected from C8-C14 fatty acids, ω-3 fatty acids, ω-6 fatty acids and polyunsaturated fatty acids.

In a preferred embodiment, the low glycemic food composition contains at least 10% by weight, preferably at least 20% by weight, at least 30% by weight or at least 40% by weight (based on total dry matter content) of a protein component.

In a preferred embodiment, the low glycemic food composition contains at least one optionally used additive. The use of the additives, depending on the field of application and profile of requirements, can be individually matched to the market and the consumer.

A preferred additive is a prebiotic. The prebiotic advantageously affects the nutritional properties of the low glycemic food composition or the functional food obtainable therefrom in a beneficial manner. The prebiotic selectively stimulates the growth and/or the activity of probiotic and/or the activity of probiotic microorganisms in the human or animal digestive tract, so that health-promoting effects occur. As prebiotic, preferably, use is made of inulin, oligofructose, resistant starch, β-glucans or galactooligo-saccharides.

A further preferred additive is a probiotic microorganism, a probiotic. These are taken to mean, in the present case, a living microbial additional component which, by stabilizing or enhancing the microbial composition in the digestive tract of the human or animal consumer, promotes the health thereof. Such microorganisms which can be used, for example, in foods, are, for example: Bifidobacterium such as the strains B. adolescentis, B. animalis, B. bifidum, B. longum, B. thermophilum; Enterococcus; Lactobacillus, such as the strains Lb. acidophilus, Lb. brevis, Lb. casei, Lb. cellobiosus, Lb. crispatus, Lb. delbrueckii subscp. Bulgaricus, Lb. fermentum, Lb. GG, Lb. johnsonii, Lb. lactis, Lb. plantarum, Lb. reuteri, Lb. rhamnosus, Lb. salivarius; Bacillus cereus toyoi; Bacillus cereus; Leuconostoc; Pediococcus acidilactici; Propionibacterium; Streptococcus such as the strains S. cremoris, S. infantarius, S. intermedius, S. lactis, S. salivarius subsp. thermophilus (cf. Fuller (1989) J. Appl. Bacteriol.). Preferred probiotics are bacteria of the genera Lactobacillus and Bifidobacterium. Such probiotic bacterial cultures can preferably be implemented as dry cultures or long-life cultures.

A further preferred additive is a symbiotic. In the present case these are taken to mean a mixture of at least one prebiotic and at least one probiotic which, by enhancement of the survival rate and increasing the number of health-promoting living microbial organisms in the gastrointestinal tract, promotes the health of the human or animal consumer, in particular by selective stimulation of the growth and/or metabolic activity of the microbial organisms.

A further preferred additive is a dairy or milk product, particularly a lactose-free milk product. Preferred dairy or milk products are skimmed-milk powder, whole milk powder, lactose-free skimmed- or whole milk powder, whey extract or whey product. It is preferably provided that the low glycemic food composition contains the milk product in an amount of 2 to 40% by weight, preferably 5 to 20% by weight (based on the total weight).

In a preferred embodiment it is provided that the low glycemic food composition contains at least one intensive sweetener, wherein the intensive sweetener is preferably selected from the group consisting of: sucralose, sodium cyclamate, acesulfame K, neohesperidin dihydrochalcone, glycyrrhizin, steveoside, monellin, thaumatin, aspartame, dulcin, saccharin, naringin dihydrochalcone, neotame and a mixture of two or more thereof.

In a preferred embodiment it is provided that the low glycemic food composition contains at least one supplement or aid which is preferably selected from the group consisting of: aroma substances such as vanillin, dyes, taste substances, emulsifiers such as lecithin, thickeners such as pectin, carob bean meal, guar gum, antioxidants, preservatives, minerals such as sodium or calcium, in particular salts such as sodium chloride, roughage, fibers, triglycerides of medium chain lengths, phytoestrogens, natural or synthetic vitamins such as vitamin A, vitamin B₁, vitamin B₂, vitamin B₃, vitamin B₅, vitamin B₆, vitamin B₁₂, vitamin B complex, vitamin C, vitamin D, vitamin E, vitamin F and vitamin K, or combinations thereof.

A supplement is taken to mean, particularly, those substances which affect the appearance, taste, organoleptic qualities, the nutritional value, nutritional properties, such as processability, storagability or readiness for use of the functional food produced.

In a preferred embodiment the functional food producible according to the invention is a medicament.

In a further preferred embodiment, the functional food producible according to the invention is a food supplement.

In a further preferred embodiment, the functional food producible according to the invention is a tube feeding formula.

Preferably, the functional food producible according to the invention is dairy produce or dairy product, in particular a cheese, butter, yogurt, kefir, quark, cultured milk, buttermilk, cream, evaporated milk, dried milk, whey, lactose, milk protein, flavored milk, low-fat milk, flavored whey or dairy fat product or preparation.

In a further preferred variant, the functional food producible according to the invention is a pudding, creme, mousse or another dessert or dessert product.

In a further preferred variant, the functional food producible according to the invention is a dairy fat product, a mixed fat product, an edible fat or an edible oil.

In a further preferred variant, the functional food producible according to the invention is a bakery product, in particular selected from bread including rolls and fine bakery products, long-life bakery products, biscuits products and wafers.

In a further preferred variant, the functional food producible according to the invention is a bread spread, in particular selected from fatty bread spreads, margarine products and baking fats.

In a further preferred variant, the functional food producible according to the invention is an instant product.

In a further preferred variant, the functional food producible according to the invention is a broth product.

In a further preferred variant, the functional food producible according to the invention is a fruit product or a fruit preparation, in particular selected from jams, marmalades, jellies, fruit preserves, fruit pulp, fruit puree, fruit juices, fruit juice concentrates, fruit nectar and fruit powders.

In a further preferred variant, the functional food producible according to the invention is a cereal product, in particular selected from muesli, muesli bars, cereal mixtures and breakfast products.

In a further preferred variant, the functional food producible according to the invention is a nonalcoholic drink, drink base or drink powder.

In a further preferred variant, the functional food producible according to the invention is a, preferably dietetic, confectionery product, in particular selected from chocolates, hard caramels, soft caramels, chewing gum, dragees, fondant products, gel products, liquorice, foamed sugar products, flakes, dragees, compactates, candied fruits, crocant, nougat products, ice sweets, marzipan and ice cream.

In a further preferred variant, the functional food producible according to the invention is an athletes and restorative food, in particular selected from protein and amino acid concentrates, whey protein hydrolyzates, isolates and concentrates, whey protein isolates and concentrates and caseinate preparations.

The present invention further relates to the use of the functional food producible according to the invention and characterized above for the treatment and/or treatment of malignant carcinomas of the human or animal body.

The present invention further relates to the use of a low glycemic food composition, food or food component for the treatment and/or prevention of malignant carcinomas of the human or animal body.

The present invention further relates to, finally, a process for the treatment and/or prevention of malignant carcinomas, wherein the process comprises at least the step: administering the functional food characterized above or a low glycemic food composition characterized above to the human or animal body in a prophylactically and/or therapeutically active amount or dose, wherein the body has at least one body cell having an activity or concentration of the enzyme TKTL-1 which is changed compared with the normal state, in particular an increased activity or concentration.

The invention will be described in more detail by the examples hereinafter, wherein the examples are not to be taken to be restrictive.

### EXAMPLES

### 1. Milk Chocolate Containing Isomaltulose (Palatinose)

COMPONENTS [%]

| | |
|---|---|
| PALATINOSE ™ | 43.00 |
| Milk powder, whole fat | 21.00 |
| Cocoa butter | 21.16 |
| Cocoa syrup | 14.30 |
| Lecithin | 0.50 |
| Vanillin | 0.02 |
| Sucralose | optional |
| TOTAL | 100.00 |

### 2. Dark Chocolate Containing Isomaltulose (Palatinose)

COMPONENTS [%]

| | |
|---|---|
| PALATINOSE ™ | 44.00 |
| Cocoa butter | 12.00 |
| Cocoa mass | 44.00 |
| Lecithin | e.g. 0.50 |
| Vanillin | e.g. 0.02 |
| TOTAL | 100.00 |

### 3. Milk Drink Containing Isomaltulose (Palatinose)

COMPONENTS [%]

| | |
|---|---|
| PALATINOSE ™ | 4.40 |
| Whole milk (fat content 3.5%) | 76.50 |
| Water | 13.80 |
| Fructose | 4.60 |
| Pectin | 0.50 |
| Yogurt culture (solution) | 0.02 |
| TOTAL | 100.00 |

### 4. Instant Drink Containing Isomaltulose (Palatinose)

### Binder:

COMPONENTS [%]

| | |
|---|---|
| Water | 11.765 |
| Vitamin mixture (Döhler) | 0.090 |
| Euroblend Apple Green (Sensient) | 0.026 |
| Sucralose | 0.025 |
| TOTAL | 11.906 |

Instant composition:

COMPONENTS [%]

| | |
|---|---|
| PALATINOSE ™ | 78.390 |
| Citric acid | 4.411 |
| Ascorbic acid | 4.411 |
| Apple aroma (Firmenich) | 0.882 |
| Binder (see above) | 11.906 |
| TOTAL | 100.00 |

1-16. (canceled)

17. A method of preventing or treating malignant carcinomas in a human or animal body, wherein said body has at least one body cell having an aerobic glucose fermentation metabolism which is altered in comparison with a normal state, and wherein the method comprises administering to a human or animal subject in need thereof an effective amount of a functional food comprised of a low glycemic food composition.

18. The method according to claim 17, wherein the low glycemic food composition has a glycemic index of less than 50.

19. The method according to claim 17, wherein the at least one body cell has an activity or concentration of transketolase-like 1 enzyme (TKTL-1) which is altered in comparison to a normal state.

20. The method according to claim 17, wherein the low glycemic food composition contains, as a carbohydrate source, at least one low glycemic carbohydrate source selected from the group consisting of monosaccharides, disaccharides, polysaccharides, monosaccharide alcohols, disaccharide alcohols and polysaccharide alcohols.

21. The method according to claim 20, wherein the low glycemic food composition contains, as a low glycemic carbohydrate source, at least one selected from the group consisting of isomaltulose, leucrose, trehalulose and turanose, alone or in combination with at least one selected from a sugar alcohol, inulin, polydextrose and resistant starch.

22. The method according to claim 20, wherein the low glycemic food composition, in addition to said low glycemic carbohydrate source, further comprises at least one high glycemic carbohydrate source.

23. The method according to claim 22, wherein the low glycemic food composition contains the low glycemic carbohydrate source and the high glycemic carbohydrate source in a weight ratio of 1:1.

24. The method according to claim 22, wherein the low glycemic food composition comprises 20% or more, by weight, of the high glycemic carbohydrate source.

25. The method according to claim 22, wherein the high glycemic carbohydrate source is selected from the group consisting of glucose-yielding disaccharides, sucrose, maltose, lactose, trehalose, maltodextrins, starch/amylose and white flour products.

26. The method according to claim 17 wherein the low glycemic food composition contains less than 2% by weight of glucose.

27. The method according to claim 17, wherein the low glycemic food composition contains at least one fat component selected from the group consisting of C8-C14 fatty acids, ω-3 fatty acids, ω-6 fatty acids and polyunsaturated fatty acids.

28. The method according to claim 17, wherein the low glycemic food composition contains a prebiotic, a probiotic, or a symbiotic.

29. The method according to claim 17, wherein the low glycemic food composition is selected from the group consisting of a medicament, a food supplement and a tube feeding formula.

30. The method according to claim 17, wherein the low glycemic food composition is selected from the group consisting of:
(i) dairy produce and dairy products;
(ii) desserts;
(iii) dairy fat products, mixed fat products; edible fats, edible oils;
(iv) bakery products;
(v) bread spreads;
(vi) instant products and broth products;
(vii) fruit products and fruit preparations;
(viii) cereal products;
(ix) non-alcoholic drinks, drink bases and drink powders;
(x) dietetic confectionery; and
(xi) athletic and restorative foods

31. The method according to claim 30, wherein said dairy produce and dairy products are selected from the group consisting of cheese, butter, yoghurt, kefir, quark, cultured milk, buttermilk, cream, evaporated milk, dried milk, whey, lactose, milk protein, flavored milk, low-fat milk, flavored whey and dairy fat products and preparations; wherein the dessert is a pudding, cream or mousse; wherein the bakery product is selected from the group consisting of bread, rolls, fine bakery products, long-life bakery products, biscuit products and wafers; wherein the bread spread is selected from the group consisting of fatty bread spreads, margarine products and baking fats; wherein the fruit products and preparations are selected from the group consisting of jams, marmalades, jellies, fruit preserves, fruit pulp, fruit puree, fruit juices, fruit juice concentrates, fruit nectar and fruit powders; wherein the cereal product is selected from the group consisting of muesli, muesli bars, cereal mixtures and breakfast products; wherein the dietetic confectionery is selected from the group consisting of chocolates, hard caramels, chewing gum, dragees, fondant products, gel products, liquorice, foamed sugar products, flakes, compactates, candied fruits, crocant, nougat products, ice sweets, marzipan and ice cream; and the athletic and restorative foods are selected from the group consisting of protein and amino acid concentrates, whey protein hydrolyzates, isolates and concentrates, whey protein isolates and concentrates, and caseinate preparations.
